(19) United States
(12) Patent Application Publication
(10) Pub. No.: US 2016/0051223 A1
(43) Pub. Date: Feb. 25, 2016

(54) DIAGNOSIS APPARATUS COMPRISING TRANSDUCER WITH VARIABLE CONFIGURATIONS AND METHOD OF MANUFACTURING THE SAME

(71) Applicant: Samsung Electronics Co., Ltd., Suwon-si (KR)

(72) Inventors: Hyungjoo KIM, Seongnam-si (KR); Yongseop YOON, Seoul (KR)

(21) Appl. No.: 14/826,729

(22) Filed: Aug. 14, 2015

(30) Foreign Application Priority Data
Aug. 20, 2014 (KR) 10-2014-0108458

(51) Int. Cl. A61B 8/00; A61B 8/08; A61B 8/14

(52) U.S. Cl. CPC A61B 8/4444; A61B 8/14; A61B 8/4477; A61B 8/0825

(57) ABSTRACT

Provided are an ultrasonic wave diagnosis apparatus including a transducer with variable configurations and a manufacturing method of the same. According to an example embodiment, an ultrasonic wave diagnosis apparatus includes a first transducer unit including a plurality of transducers and a second transducer unit including a plurality of transducers. The first transducer unit and the second transducer unit may be symmetrically placed about a subject. The first transducer unit and the second transducer unit may be connected to each other in a sliding manner.

### RELATED APPLICATION

This application claims the benefit of Korean Patent Application No. 10-2014-0108458, filed on Aug. 20, 2014, in the Korean Intellectual Property Office, the disclosure of which is incorporated herein in its entirety by reference.

### BACKGROUND

1. Field

The present disclosure relates to a diagnosis apparatus including a transducer with variable configurations and a method of manufacturing the same.

2. Description of the Related Art

Ultrasonic wave tomography is performed using an imaging apparatus that generally includes an ultrasonic wave transducer placed around an object. A probe is used to scan sections of an object to produce an entire image of the object. For example, the object may be a breast. During scanning, the ultrasonic wave transducer delivers an ultrasonic signal to the breast.

In general, the transducer moves vertically to scan the entire breast. Also, the transducer scans the breast by mechanically rotating around the breast or by using an electric switching method depending on the structure of the transducer. Thus, ultrasonic signals that are generated by the transducer in multiple directions with respect to the breast are recorded.

However, when the transducer mechanically rotates around the breast, the movement of the transducer causes a vortex in a liquid, which may affect the signal corresponding to the ultrasonic wave. Additionally, the breast may move due to the vortex, thereby affecting the image of the breast during scanning.

A circular-shape transducer formed as a single body may be placed to fully surround the breast and transmit and receive ultrasonic waves in every direction with respect to the breast by using an electric switching method in which no mechanical rotation of the transducer takes place. Thus, the occurrence of a vortex in a liquid may be prevented.

However, if the transducer is formed as a single body as described above, the diameter of the transducer is fixed and thus the diameter cannot be adjusted. Accordingly, a focal length in an elevation direction is also fixed.

In contrast, the breast size is different according to each person. Also, the breast diameter defined as the length from the bottom of the breast to the top of the breast varies with each person. Thus, it may be difficult to adjust a focal length of an existing circular-shape transducer formed as a single-body in accordance with the breast size of a subject to be examined.

### SUMMARY

Provided is a diagnosis apparatus including a transducer with variable configurations that may shorten testing time and produce more accurate images by preventing degradation in the quality of subject images.

Provided is a manufacturing method of the diagnosis apparatus.

Additional features will be set forth in part in the description which follows and, in part, will be apparent from the description, or may be learned by practice of the example embodiments.

According to some of the example embodiments, an ultrasonic wave diagnosis apparatus may include a first transducer unit including a plurality of transducers, the first transducer unit may be configured to emit ultrasonic waves towards a subject and a second transducer unit including a plurality of transducers, the second transducer unit may be configured to receive the ultrasonic waves emitted by the first transducer unit.

According to an example embodiment, the first transducer unit and the second transducer unit may be configured to be symmetrically disposed around the subject.

According to an example embodiment, the first transducer unit and the second transducer unit may be configured to connect to each other. Additionally, the first transducer unit and the second transducer unit may be configured to connect in a sliding manner.

According to an example embodiment, the first transducer unit may include a first transducer that may be configured to emit first ultrasonic waves towards the subject, a second transducer that may be configured to emit second ultrasonic waves towards the subject, and a connecting member that may be configured to connect the first and second transducers.

According to an example embodiment, the second transducer unit may include a first transducer that may be configured to receive the ultrasonic waves emitted by the first transducer unit, a second transducer that may be configured to receive the ultrasonic waves emitted by the first transducer unit, and a connecting member that may be configured to connect the first and second transducers.

According to some of the example embodiments, a method of manufacturing an ultrasonic wave diagnosis apparatus may include preparing a first transducer that may include a first ultrasonic wave transmitter/receiver and a first connector, preparing a second transducer that may include a second ultrasonic wave transmitter/receiver and a second connector, and connecting the first and second connectors, thereby forming a first transducer unit of a ultrasonic wave diagnosis apparatus.

According to an example embodiment, the method may further include preparing a third transducer that may include a third ultrasonic wave transmitter/receiver and a third connector, preparing a fourth transducer that may include a fourth ultrasonic wave transmitter/receiver and a fourth connector, and connecting the third and fourth connectors, thereby forming a second transducer unit of the ultrasonic wave diagnosis apparatus.

According to an example embodiment, the preparing of the first transducer may include forming a first through hole in the first connector.

According to an example embodiment, the preparing of the second transducer may include forming a second through hole in the second connector.

According to an example embodiment, the connecting of the first and second connectors may include inserting a connecting member, which passes through the first and second connectors, into the first and second connectors.

According to an example embodiment, the preparing of the third transducer may include forming a third through hole in the third connector.

According to an example embodiment, the preparing of the fourth transducer may include forming a fourth through hole in the fourth connector.

According to an example embodiment, the connecting of the third and fourth connectors may include inserting a connecting member, which passes through the third and fourth connectors, into the third and fourth connectors.

According to an example embodiment, the first transducer may further include a first sliding portion, and the third transducer may further include a second sliding portion.

According to an example embodiment, elements required for connecting the first sliding portion to the second sliding portion may be formed to the first sliding portion before connecting the first and second transducers to each other, and elements needed for the connection with the first sliding portion may be formed to the second sliding portion before connecting the third and fourth transducers to each other.

According to an example embodiment, the first sliding portion and the second sliding portion may be connected after the first and second transducers are connected to each other and after the third and fourth transducers are connected to each other.

According to an example embodiment, the first sliding portion and the second sliding portion may be connected to each other, and then the first and second transducers are connected to each other and the third and fourth transducers are connected to each other.

### BRIEF DESCRIPTION OF THE DRAWINGS

The foregoing and other features of some example embodiments will be apparent from the more particular description of non-limiting embodiments of some example embodiments, as illustrated in the accompanying drawings in which like reference characters refer to like parts throughout the different views. The drawings are not necessarily to scale, emphasis instead being placed upon illustrating principles of some example embodiments. In the drawings:

FIG. 1 is a plan view of a variable-shape ultrasonic wave transducer according to an example embodiment;

FIG. 2 is a plan view showing one-to-one correspondence of transmitters and receivers in the transducer of FIG. 1, according to an example embodiment;

FIG. 3 is a plan view showing a different way of transmitting and receiving an ultrasonic wave, compared with FIGS. 1 and 2, according to an example embodiment;

FIG. 4 is a perspective view of the transducer of FIG. 1, according to an example embodiment;

FIG. 5 is a plan view showing dispositions of first to fourth transducers when a subject is bigger than that of FIG. 1, according to an example embodiment;

FIG. 6 is a plan view of a transducer according to another example embodiment;

FIG. 7 is a cross-sectional view of an example of first and second sliding portions taken a long line of 7-7′ of FIG. 6, according to an example embodiment;

FIG. 8 is a plan view of the first sliding portion of FIG. 7, according to an example embodiment;

FIG. 9 is a cross-sectional view of another example of first and second sliding portions taken a long line of 7-7′ of FIG. 6, according to an example embodiment;

FIG. 10 is a cross-sectional view taken along line 10-10′ of FIG. 9, according to an example embodiment;

FIG. 11 is a cross-sectional view of a medical diagnosis apparatus according to another example embodiment;

FIG. 12 is a cross-sectional view showing a case in which the medical diagnosis apparatus of FIG. 11 maintains a constant depth of focus for an ultrasonic wave as the size of the subject changes, according to an example embodiment;

FIG. 13 is a block diagram of an ultrasonic imaging system including a transducer unit according to an example embodiment;

FIG. 14 is a flow chart showing a method of scanning a subject by using a transducer according to an example embodiment; and

FIGS. 15 and 16 are cross-sectional views that show a manufacturing method of a transducer according to another example embodiment.

### DETAILED DESCRIPTION

Example embodiments will now be described more fully with reference to the accompanying drawings, in which some example embodiments are shown. Example embodiments, may, however, be embodied in many different forms and should not be construed as being limited to the embodiments set forth herein; rather, these example embodiments are provided so that this disclosure will be thorough and complete, and will fully convey the scope of example embodiments to those of ordinary skill in the art. In the drawings, the thicknesses of layers and regions are exaggerated for clarity. Like reference characters and/or numerals in the drawings denote like elements, and thus their description may be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein the term “and/or” includes any and all combinations of one or more of the associated listed items.

It will be understood that, although the terms “first”, “second”, etc. may be used herein to describe various elements, components, regions, layers and/or sections. These elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof. Expressions such as “at least one of,” when preceding a list of elements, modify the entire list of elements and do not modify the individual elements of the list.

Example embodiments are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

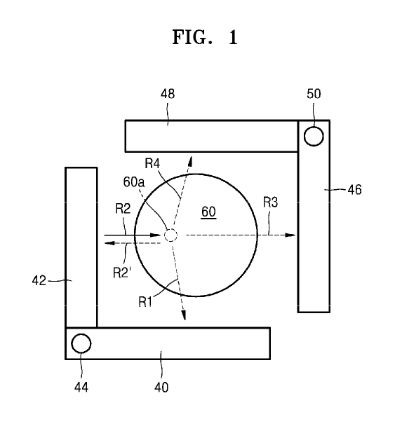

FIG. 1 is a plan view of a variable-shape ultrasonic wave transducer according to an example embodiment.

Referring to FIG. 1, the variable-shape ultrasonic wave transducer, that is, an ultrasonic wave transducer with variable configurations may include first to fourth transducers 40, 42, 46, and 48. However, the example embodiments are not limited thereto, and the variable-shape ultrasonic wave transducer may include more or less transducers than those shown in FIG. 1. Each transducer may emit an ultra-high frequency sound wave, typically between 1 to 18 MHz, directed towards a subject for diagnostic imaging purposes, including in the medical, pharmaceutical, scientific, military, robotics, ecological, agricultural, geological, and industrial fields. Each of the transducers may also receive the return of a previously emitted ultra-high frequency sound wave and convert the received sound wave into an electrical signal. The electrical signal may be transmitted to a ultrasonic wave imaging device that may provide for display a two-dimensional (2D) or three-dimensional (3D) image based on the received electrical signals. The first and second transducers 40 and 42 may be connected to each other, and the third and fourth transducers 46 and 48 may be connected to each other. The first and second transducers 40 and 42 may be spaced apart from the third and fourth transducers 46 and 48. The first to fourth transducer 40, 42, 46 and 48 may be symmetrically disposed about a subject 60. For example, the first and second transducers 40 and 42 and the third and fourth transducers 46 and 48 may be symmetrically disposed about the subject 60. An end of the first transducer 40 and an end of the second transducer 42 may be connected to each other through a first connecting member 44. The first and second transducers 40 and 42 may rotate around the first connecting member 44 using the first connecting member 44 as an axis of rotation. Accordingly, an angle between the first transducer 40 and the second transducer 42 may be adjusted. For example, the angle between the first transducer 40 and the second transducer 42 may be 90 degrees or more, or 90 degrees or less. The angle between the first transducer 40 and the second transducer 42 may be adjusted in real time according to the size of the subject 60, using a controller that may control the operation of the transducers.

A first connecting member 44 may be, for example, a hinge, an actuator, or the like. The connecting member 44 may receive signals from a controller that may provide control commands that set the angle that the first transducer 40 and second transducer 42 form. The length of the first transducer 40 and the length of the second transducer 42 may be identical to or different from each other. Each of the first and second transducers 40 and 42 may transmit and receive an ultrasonic wave. In this regard, as shown in FIG. 1, the second transducer 42 may be, for example, a transmitter that may transmit an ultrasonic wave toward the subject 60, and the other transducers 40, 46 and 48 may be receivers that may receive the ultrasonic wave that has passed through the subject 60. Alternatively, the first transducer 40 may be a transmitter, and the other transducers 42, 46 and 48 may be receivers. Also, the third and fourth transducers 46 and 48 may serve as transmitters, and the first and second transducers 40 and 42 may serve as receivers. A transducer may be capable of transmitting and receiving an ultrasonic wave, and thus when any one or two of the first to fourth transducers 40, 42, 46 and 48 serve as transmitters, the others may serve as receivers.

An end of the third transducer 46 and an end of the fourth transducer 48 may be connected by a second connecting member 50. The third and fourth transducers 46 and 48 may rotate around the second connecting member 50 using the second connecting member 50 as a rotation axis. Accordingly, an angle between the third transducer 46 and the fourth transducer 48 may be adjusted when needed. The second connecting member 50 and the first connecting member 44 may be identical to or different from each other. The length of the third transducer 46 and the length of the fourth transducer 48 may be identical to or different from each other. In a case in which the size of the subject 60 is smaller than an area defined by the first to fourth transducers 40, 42, 46 and 48, the first transducer 40 and the fourth transducer 48 may be disposed in parallel while facing each other, with the subject 60 in between. In a case in which the size of the subject 60 is not big, the second transducer 42 and the third transducer 46 may be disposed in parallel while facing each other, with the subject 60 in between. The subject 60 may be a part of a body, for example, an arm, a leg, or a breast of a woman.

In FIG. 1, a solid arrow line R2 indicates an ultrasonic wave transmitted from the second transducer 42 toward a first area 60a of the subject 60. The ultrasonic wave R2 transmitted toward the first area 60a may be scattered in various directions, thereby causing an ultrasonic wave R1 to be incident on the first transducer 40, an ultrasonic wave R3 to be incident on the third transducer 46, and an ultrasonic wave R4 to be incident on the fourth transducer 48. The scattered ultrasonic waves R1, R3, and R4 may include information about the subject 60. By receiving the scattered ultrasonic waves R1, R3, and R4 that passed through the subject 60, the information about an inner organism of the subject 60 may be obtained. Meanwhile, after the second transducer 42 transmits the ultrasonic wave R2 to the first area 60a of the subject 60, an ultrasonic wave R2′, which is from the first area 60a, may be generated. The reflected ultrasonic wave R2′ may also include information about an inner organism of the subject 60. The reflected ultrasonic wave R2′ may be incident on the second transducer 42. In this regard, the second transducer 42 may not only transmit the ultrasonic wave R2 but may also receive the reflected ultrasonic wave R2′. The other transducers 40, 46, and 48 may also function similar to the second transducer 42. For example, the first transducer 40 may transmit an ultrasonic wave toward the first area 60a (or another area) of the subject 60, and the other transducers 42, 46, and 48 may receive ultrasonic waves resulting from the scattering of the ultrasonic wave transmitted by the first transducer 40. In this case, the first transducer 40 may receive the reflected ultrasonic wave from the first area 60a. As described in the above examples, by sequentially using the transducers 40, 42, 46, and 48 as transmitters and receivers, the transducers 40, 42, 46, and 48 may transmit an ultrasonic wave toward the subject 60 and may receive the ultrasonic wave from the subject 60, thereby obtaining more accurate information (e.g. a more accurate image) about the inner state of the subject 60. Filtering and preprocessing are performed for signals transmitted by each of the first to fourth transducers 40, 42, 46, and 48, which respectively are the scattered ultrasonic waves R1, R3, and R4, and the reflected ultrasonic wave R2′, in order to obtain an image about the inner state of the subject 60, and then, an image reconstruction process is performed by using a software algorithm. During the aforementioned process, the first to fourth transducers 40, 42, 46, and 48 do not rotate, and thus the problems of the existing transducers do not occur.

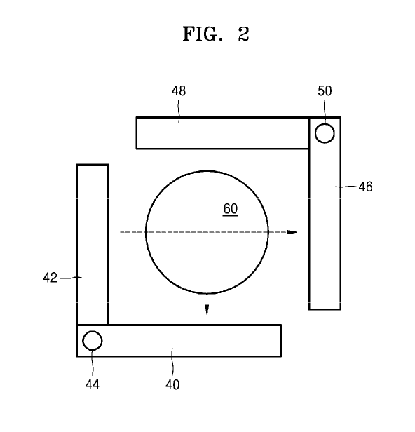

FIG. 2 is a plan view according to an example embodiment showing a one-to-one correspondence of transmitters and receivers in the transducer of FIG. 1.

Referring to FIG. 2, the second and the fourth transducers 42 and 48 may be transmitters that may transmit ultrasonic waves, the first transducer 40 may be a receiver that may receive the ultrasonic wave transmitted from the fourth transducer 48, and the third transducer 46 may be a receiver that may receive the ultrasonic wave transmitted from the second transducer 42.

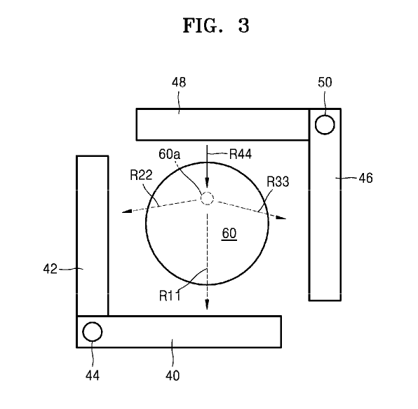

FIG. 3 is a plan view according to an example embodiment showing a different way of transmitting and receiving an ultrasonic wave, in comparison with FIGS. 1 and 2.

Referring to FIG. 3, the fourth transducer 48 may be a transmitter that may transmit an ultrasonic wave R44 to the first area 60a of the subject 60, and the transducers 40, 42, and 46 may be receivers that may receive the scattered ultrasonic waves R11, R22, and R33 from the first area 60a. The fourth transducer 48 may also receive the reflected ultrasonic wave from the first area 60a of the subject 60.

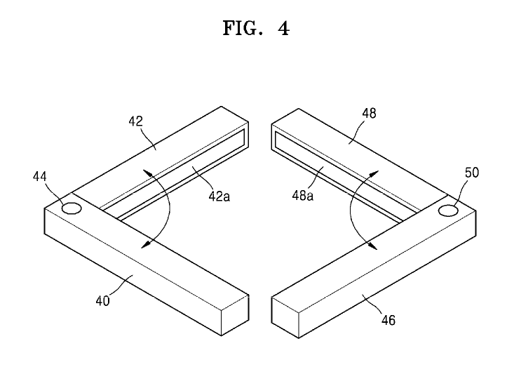

FIG. 4 is a perspective view of the transducer of FIG. 1 according to an example embodiment. The subject is omitted for the sake of convenience in FIG. 4.

Referring to FIG. 4, the second transducer 42 may have a window 42a for transmitting and receiving an ultrasonic wave. The fourth transducer 48 may also have a window 48a for transmitting and receiving an ultrasonic wave. Although not shown in the perspective view of FIG. 4, the first and third transducers 40 and 46 may also have windows for transmitting and receiving an ultrasonic wave, located on surfaces that respectively face the fourth transducer 48 and the second transducer 42.

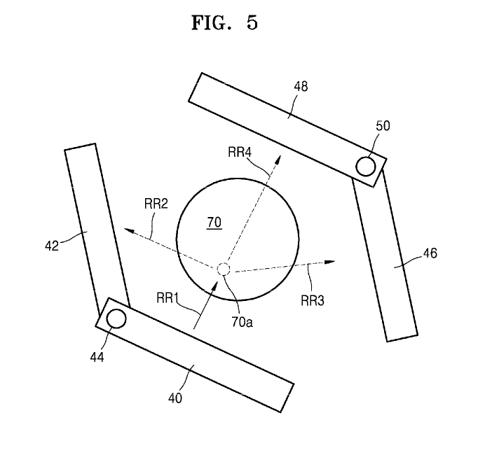

FIG. 5 is a plan view according to an example embodiment showing dispositions of the first to fourth transducers 40, 42, 46, and 48 when a subject is bigger than that of FIG. 1.

Referring to FIG. 5, if a subject 70 is bigger than the subject 60 of FIG. 1, the boundary of the subject 70 may extend beyond an effective area (service area) of an ultrasonic wave test that may be performed by the first to fourth transducers 40, 42, 46, and 48 that are disposed as illustrated in FIG. 1. In this case, the angle between the first and second transducers 40 and 42 and the angle between the third and fourth transducers 46 and 48 may be greater than 90 degrees. In addition, the angle between the first and second transducers 40 and 42 may be identical to or different from the angle between the third and fourth transducers 46 and 48. Even in a case in which the angle between the first and second transducers 40 and 42 and the angle between the third and fourth transducers 46 and 48 are greater than 90 degrees, the first to fourth transducers 40, 42, 46, and 48 may be disposed symmetrically. In FIG. 5, RR1 indicates an ultrasonic wave that is transmitted from the first transducer 40 to the first area 70a of the subject 70. Each of RR2, RR3, and RR4 indicates an ultrasonic wave that is incident to each of the second, third, and fourth transducers 42, 46, and 48 after being scattered at the first area 70a. In FIG. 5, the first transducer 40 may be selected as an ultrasonic wave transmitter, but the other transducers 42, 46, and 48 may also be selected as ultrasonic wave transmitters. The other transducers except for the transducer selected as a transmitter may serve as receivers that may receive the ultrasonic waves that come via the subject 70. The subject 70 may be the same kind as the subject 60 of FIG. 1.

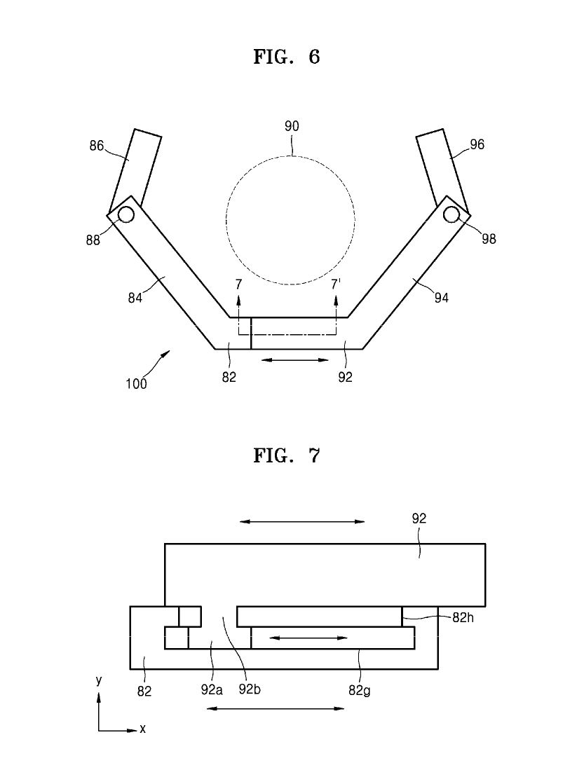

FIG. 6 is a plan view of a transducer according to another example embodiment.

Referring to FIG. 6, a transducer 100 according to another example embodiment may include a first to fourth transducers 84, 86, 94, and 96. An end of the first transducer 84 may be connected to a first sliding portion 82. The first transducer 84 and the first sliding portion 82 may be formed as a single body without a connecting portion. In this case, when the first sliding portion 82 moves, the first transducer 84 and a second transducer 86 connected to the first sliding portion 82 may move together. The other end of the first transducer 84 may be connected to one end of the second transducer 86. The first transducer 84 and the second transducer 86 may be connected to each other by a first connecting member 88. The second transducer 86 may rotate around the first connecting member 88 using the first connecting member 88 as a rotation axis, and the degree of rotation may be adjusted based on the size of a subject 90. The first connecting member 88 may be a hinge or an actuator that passes through the first and second transducers 84 and 86.

An end of a third transducer 94 may be connected to a second sliding portion 92. The third transducer 94 may be formed as a single body with the second sliding portion 92. Therefore, when the second sliding portion 92 moves, the third transducer 94 and a fourth transducer 96 connected thereto may move together. The second sliding portion 92 and the first sliding portion 82 may be connected to each other in a sliding manner. The other end of the third transducer 94 may be connected to one end of the fourth transducer 96. The third transducer 94 and the fourth transducer 96 may be connected by a second connecting member 98. The fourth transducer 96 may rotate around the second connecting member 98 using the second connecting member 98 as a rotation axis, and the degree of rotation may be adjusted based on the size of the subject 90. The second connecting member 98 may be, for example, a hinge, an actuator, or the like that passes through the third and fourth transducers 94 and 96. The first and second connecting member 88 and 98 may have various forms and be of various types that may rotatably connect two transducers. A connecting member by which two objects may be rotatably connected to each other is well known to those skilled in the art. Thus the detailed description about the connecting member is omitted herein.

The first and second sliding portions 82 and 92 may move relative to each other. For example, when the first sliding portion 82 is fixed, the second sliding portion 92 along with the third and fourth transducers 94 and 96 may be moved toward or away from the first sliding portion 82 by moving the second sliding portion 92 forward or backward. On the other hand, if the second sliding portion 92 is fixed, the first sliding portion 82 along with the first and second transducers 84 and 86 may be spaced apart from or get close to the second sliding portion 92 by moving the first sliding portion 82. In addition, the first and second sliding portions 82 and 92 may move together so that the transducers move toward or away from one another. These movements may be determined according to the size of the subject 90.

When transmitting and receiving an ultrasonic wave, the first transducer 84 may be selected as a first ultrasonic wave transmitter. The first transducer 84 may transmit an ultrasonic wave toward the subject 90, and the ultrasonic wave may be incident on the second to the fourth transducers 86, 94, and 96 via the subject 90. The first transducer 84 may also receive the ultrasonic wave reflected from the subject 90. When the ultrasonic wave radiation by the first transducer 84 is finished, one of the second to the fourth transducers 86, 94, and 96, may be selected as a second ultrasonic wave transmitter. The second ultrasonic wave transmitter may transmit an ultrasonic wave toward the subject 90, and the other transducers may receive the ultrasonic waves that come via the subject 90. As described in the above example, by sequentially using the first to fourth transducers 84, 86, 94, and 96 as ultrasonic wave transmitters, information (e.g. an image) about the subject 90 may be obtained in various directions and a more accurate analysis of the subject 90 may be conducted. The same method applies to the transducers in FIGS. 1 and 5. The subject 90 may be identical to the subject 60 of FIG. 1.

Meanwhile, although four transducers 84, 86, 94, and 96 are illustrated in FIG. 6, a multi-jointed transducer having five or more transducers may be provided. For example, one transducer may further be connected to the second transducer 86, and one transducer may further be connected to the fourth transducer 96. These added transducers may be rotatably connected to the second and the fourth transducers 86 and 96.

FIG. 7 is a cross-sectional view of an example of first and second sliding portions 82 and 92 taken along line of 7-7′ of FIG. 6 according to an example embodiment.

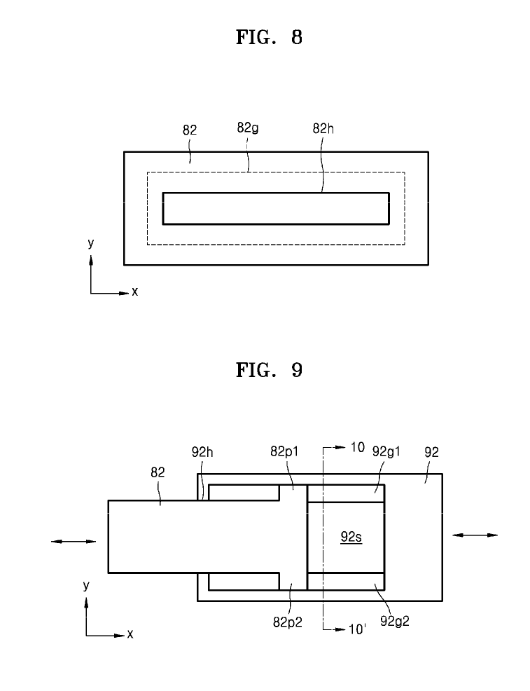

Referring to FIG. 7, a space 82g exists inside the first sliding portion 82. The space 82g may have a desired (and/or predetermined) height and length. The length (x-axis direction) of the space 82g may be greater than or equal to the height (y-axis direction) thereof. The space 82g may be used as a movement space for the second sliding portion 92. A through hole 82h may be formed on the upper portion of space 82g of the first sliding portion 82. As shown in FIG. 8, which is a plan view of the first sliding portion of FIG. 7, the through hole 82h may be rectangular shaped and may have a longer side in an x-axis direction. The through hole 82h may be connected to the space 82g. Accordingly, the through hole 82h and the space 82g as a whole may be a movement space for the second sliding portion 92. The second sliding portion 92 may have projecting portions 92a and 92b on the bottom surface thereof. The projecting portions 92a and 92b may be formed in an inverse ‘T’ shape. The projecting portions 92a and 92b may include a wide projecting portion 92a and a narrow projecting portion 92b. The wide projecting portion 92a may be placed within the space 82g of the first sliding portion 82 and may move within the space 82g. The narrow projecting portion 92b may be placed in the through hole 82h and may move within the space defined by the through hole 82h.

FIG. 8 is a plan view of the first sliding portion 82 of FIG. 7 according to an example embodiment.

Referring to FIG. 8, the through hole 82h and the space 82g may be formed lengthwise in an x-axis direction. The length and the height of the space 82g in the x-axis and the y-axis directions may be greater than those of the through hole 82h. The through hole 82h may be placed within the space 82g.

FIG. 9 is a cross-sectional view of another example embodiment of first and second sliding portions 82 and 92 taken along line of 7-7′ of FIG. 6.

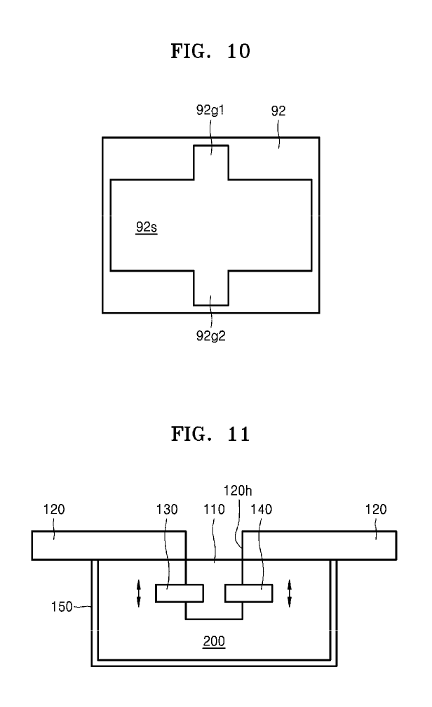

Referring to FIG. 9, the second sliding portion 92 may include a space 92s and first and second grooves 92g1 and 92g2 inside thereof. The space 92s and the first and second grooves 92g1 and 92g2 may have a desired (and/or predetermined) length in an x-axis direction and a desired (and/or predetermined) width in a y-axis direction. The lengths of the space 92s and the first and second grooves 92g1 and 92g2 in the x-axis direction may be identical to or different from one another. The width of the space 92s in the y-axis direction may be identical to or different from those of the first and second grooves 92g1 and 92g2. As shown in FIG. 10, the first groove 92g1 may be formed on the upper portion of the space 92s, and the second groove 92g2 may be formed on the lower portion of the space 92s. The space 92s and the first and second grooves 92g1 and 92g2 may be connected to one another. The second sliding portion 92 may have a through hole 92h at the end portion in a −x-axis direction. The space 92s may be exposed through the through hole 92h. A diameter of the through hole 92h may be identical to or different from the width of the space 92s in the y-axis direction. The first sliding portion 82 may be connected to the second sliding portion 92 via the through hole 92h of the second sliding portion 92. The first sliding portion 82 may have first and second projecting portions 82p1 and 82p2 at the end portion thereof in the x-axis direction. The first projecting portion 82p1 may protrude in a y-axis direction whereas the second projecting portion 82p2 may protrude in a −y-axis direction. Portion of the first sliding portion 82 may be placed within the space 92s of the second sliding portion 92 via the through hole 92h. The first sliding portion 82 may move in the x-axis or the −x-axis directions within the space 92s. Alternatively, the second sliding portion 92 may move in the x-axis or the −x-axis directions. The first projecting portion 82p1 may be placed in the first groove 92g1, and the second projecting portion 82p2 may be placed in the second groove 92g2.

FIG. 11 is a cross-sectional view of a medical diagnosis apparatus according to another example embodiment.

Referring to FIG. 11, a case 150 may be provided under a bed 120. The bed 120 and the case 150 may be sealed together. The case 150 may be filled with a liquid 200. The liquid 200 may be, for example, water. A through hole 120h may be formed in the bed 120. Although only one through hole 120h is illustrated for the sake of convenience, the bed 120 may have another through hole in addition to the through hole 120h. A subject 110 may be placed in the liquid 200 in the case 150 via the through hole 120h. The subject 110 may be a part of a body, for example, a breast of woman, an ankle, a wrist, a leg, a thigh, or the like. For the sake of convenience, the subject 110 may be quadrangular shaped in FIG. 11. In a case in which the subject 110 is a breast of a woman, the woman may lay face down on the bed 120 and thus the breast of the woman may be positioned via the through hole 120h of the bed 120. A first transducer unit 130 and the second transducer unit 140 may be disposed at the circumference of the subject 110. Each of the first and second transducer units 130 and 140 may include a plurality of transducers. The first transducer unit 130 may include, for example, the first and second transducers 40 and 42 of FIG. 1. The second transducer unit 140 may include, for example, the third and fourth transducers 46 and 48 of FIG. 1. Also, the first and second transducer units 130 and 140 may be a multi-jointed transducer such as the transducer 100 of FIG. 6 in which multiple transducers may be connected to each other. The first and second transducer units 130 and 140 and the subject 110 may be spaced apart from one another, and the liquid 200 may exist in between.

The first and second transducer units 130 and 140, as illustrated in FIG. 1 or FIG. 6, may be disposed at the circumference of the subject 110 by connecting the plurality of transducers through the connecting member. In this regard, the first and second transducer units 130 and 140 may transmit an ultrasonic wave toward the subject 100 in various directions without rotating around the subject 110. In other words, during a test, the first and second transducer units 130 and 140 do not mechanically rotate around the subject 110. In a conventional way of testing, a transducer mechanically rotates around a subject, thereby generating a vortex in a liquid. Such a vortex in the liquid causes a subject to move, and thus the quality of the inner image of the subject is degraded. By using the transducer described herein, the aforementioned problem may be solved. In addition, the first and second transducer units 130 and 140 may conduct the test by moving upward and downward alongside the subject 110 without a mechanical rotation, and the time for the test may be less than that of the conventional test. When the first and second transducer units 130 and 140 are used for a test, ultrasonic waves may be transmitted and received in multiple directions, and thus more information about the subject 110 may be obtained compared to conventional tests. As a result, a more accurate image about the subject 110 may be produced compared to the conventional tests.

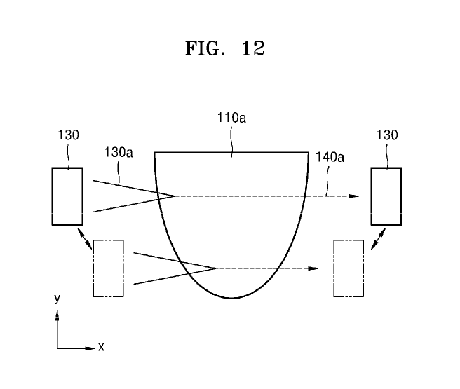

Meanwhile, portions of the subject 110 may vary in size. For example, in a case in which the subject 110 is a breast 110a of a woman as shown in FIG. 12, the diameter of the breast 110a in an x-axis direction may vary along a y-axis direction. In this case, as the first and second transducer units 130 and 140 move along the y-axis, the distance between the first and second transducer units 130 and 140 may change in real-time. That is, the distance between the first and second transducer units 130 and 140 may be controlled in real time. Also, an angle between the transducers (e.g. the first and second transducers 40 and 42 in FIG. 1) included in the first transducer unit 130 may be adjusted by a controller in response to changes in the size of the subject 110, and an angle between the transducers (e.g. the third and fourth transducers 46 and 48 in FIG. 1) included in the second transducer unit 140 may also be adjusted by a controller.

In this regard, as shown in FIG. 12, when the ultrasonic wave is transmitted from the first transducer unit 130 toward the breast 110a, a point (depth of focus) to which the ultrasonic wave is radiated may be maintained with a constant distance from the surface of the breast even when the breast 110a vary in size, that is, even when the first transducer unit 130 moves in the y-axis direction. When using the transducer described herein, the transducer effectively responds to changes in the size of the subject, and thus the transducer may keep the depth of focus of an ultrasonic wave constant regardless of changes in the size of the subject.

A reference numeral 130a in FIG. 12 according to an example embodiment refers to an ultrasonic wave transmitted from the first transducer unit 130, and a reference numeral 140a refers to an ultrasonic wave that passes through the breast 110a and is received by the second transducer unit 140.

While references have been made herein regarding the use of the example embodiments in relation to medical imaging, specifically medical imaging of a woman's breast, the example embodiments are not limited thereto and may also be used in connection with other technical fields, including for pharmaceutical purposes, industrial purposes, ecological purposes, geological purposes, agricultural purposes, scientific purposes, military purposes, robotic purposes, etc.

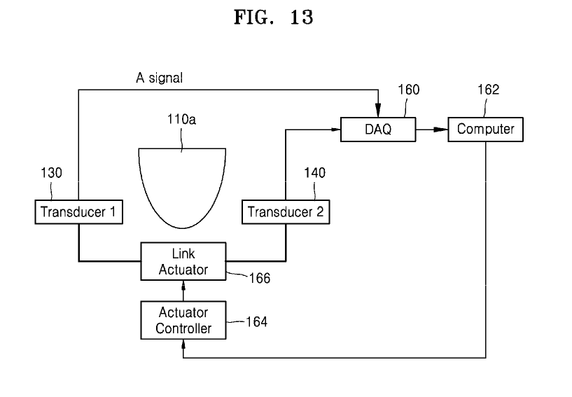

FIG. 13 is a block diagram of an ultrasonic imaging system including a transducer unit according to an example embodiment. The ultrasonic imaging system illustrated in FIG. 13 may be included in the medical diagnosis apparatus of FIG. 11.

Real-time control of the first and second transducer units 130 and 140 in accordance with changes in the size of the subject, i.e., real-time control of operations of transducers in the first and second transducer units 130 and 140 is described with reference to FIG. 13.

Referring to FIG. 13, data of a signal generated from the first transducer unit 130 and the second transducer unit 140 may be obtained by a data acquisition system, (DAQ) 160. The signal may include a signal corresponding to a ultrasonic wave that passes through the breast 110a and a signal corresponding to a ultrasonic wave reflected from the breast 110a. Data obtained via the DAQ 160 may be transmitted to a computer 162. Based on the transmitted data, the computer 162 may obtain distance information about the distance between the breast 110a and each of the first and second transducer units 130 and 140. The distance information is compared with distance information desired (and/or pre-set) by a user, and then the computer 162 transmits a signal to an actuator controller 164 to adjust or maintain the distance between the breast 110a and each of the first and second transducer units 130 and 140 according to the distance information desired (and/or pre-set) by the user.

Based on the signal, the actuator controller 164 may control operations of a link actuator 166 configured to directly control operations of the first and second transducer units 130 and 140. In this regard, the distance between the breast 110a and each of the first and second transducer units 130 and 140 may be adjusted or maintained according to the distance information desired (and/or pre-set) by a user. The distance set by the user may be determined based on a size of the breast 110a and a focal length of an ultrasonic wave.

A process of transmitting data to the computer 162 and a process of transmitting data from the computer 162 to the actuator controller 164 are performed in real-time. Accordingly, operations of the first and second transducer units 130 and 140 may be controlled by the link actuator 166 in real-time during an ultrasonic wave diagnosis. In this regard, operations of transducers included in the first transducer unit 130 (e.g., the first and second transducers 40 and 42 of FIG. 1) and transducers included in the second transducer unit 140 (e.g., the third and fourth transducers 46 and 48 of FIG. 1) may also be controlled in real-time.

Operations of the first and second transducer units 130 and 140 may be controlled in real-time, and thus, as described with reference to FIG. 12, when a size of the breast 110a changes, an angle between the transducers included in the first and second transducer units 130 and 140 may be controlled in real-time to adjust the distance between the subject 110 and each of the first and second transducer units 130 and 140 according to the distance information desired (and/or pre-set) by the user.

Data transmitted to the computer 162 via the DAQ 160 may include image data about an inner area of the breast 110a. In this case, an image reconstruction process may be performed based on the image data described in connection with FIG. 1. The image reconstruction process may be performed by the computer 162 or by a separate and independent image reconstruction apparatus connected to the computer 162. The reconstructed image may be displayed via the computer 162 or via a separate and independent display apparatus.

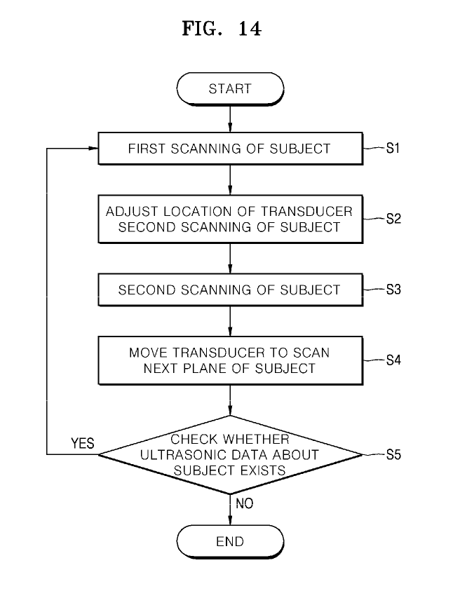

A method of scanning a subject by using a transducer according to example embodiment will be described below with reference to FIG. 14.

Referring to FIG. 14, a first scanning of a subject is performed (S1). Geometric data of the subject may be obtained by performing the first scanning. The geometric data may include, for example, a location of the subject, an outer shape of a part of the subject, or the like.

To perform the first scanning, an ultrasonic wave is emitted from an ultrasonic wave transmitter of a selected transducer (e.g., the second transducer 42 of FIG. 1) towards the subject. A part of the ultrasonic wave may be reflected by a boundary surface between the subject and a material surrounding the subject (e.g., water), and the reflected ultrasonic wave may be received by the selected transducer. By using the reflected ultrasonic wave, the distance between the selected transducer and the subject, which is distance information between the subject and a transducer unit around the subject, may be obtained. The obtained location information is compared with the distance, which is set by a user, between the subject and the transducer unit, and then the location of the transducer is adjusted according to the distance set by the user for a second scanning of the subject (S2). The distance set by the user may be determined taking into account the size of the subject and the focal length of the emitted ultrasonic wave. After the location of the transducer is adjusted, the second scanning of the subject is performed (S3). By performing the second scanning, an image of an inner area of the subject may be obtained, and the aforementioned process may be performed as described in FIG. 13. After performing the second scanning, a location of the transducer, i.e., a location of the transducer unit is changed in order to scan a next plane of the subject (S4). For example, when the subject is the breast 110a of FIG. 12, the first and second transducer units 130 and 140 may be moved upward or downward along a surface of the breast 110a. After the location of the transducer is changed, an operation S5 that checks whether ultrasonic wave data about the subject exists is performed. In operation S5, it is checked whether the transducer is within a region where the subject exists. That is, in operation S5, it is checked whether the transducer is out of a scanning region of the subject. In operation S5, when the ultrasonic wave data about the subject exists (Y), operations S1 to S4 are repeated. Every time the operations are repeated, new geometric data about the subject with respect to the moved location of the transducer is obtained, and a new scanning of the subject is performed based on the newly obtained geometric data. Accordingly, by repeating these processes, changes in the size of the subject may be detected in real-time, and the distance between the transducer and the subject may be newly set in accordance with the changes in the size of the subject. In operation S5, the ultrasonic wave data about the subject does not exist (N), the scanning of the subject is terminated.

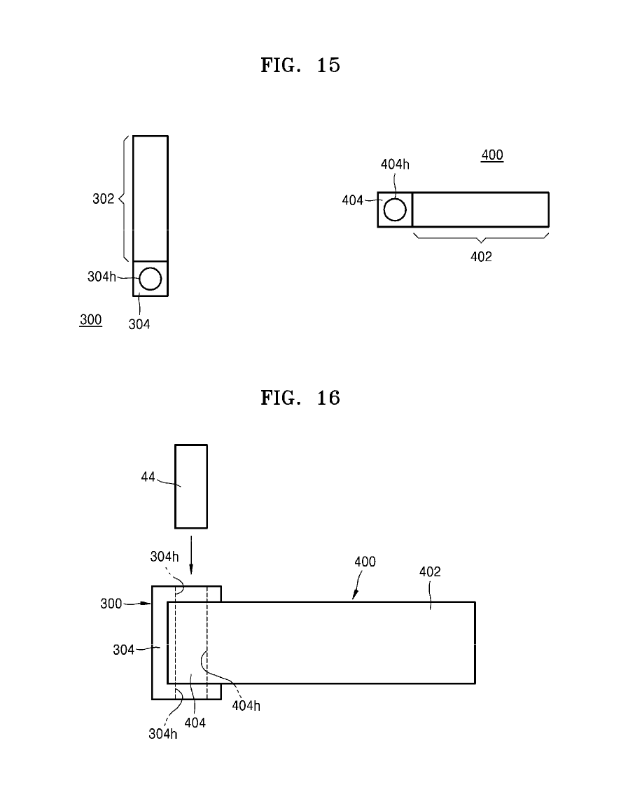

Hereinafter, a method of manufacturing a transducer according to an example embodiment will be described in detail by referring to FIGS. 15 and 16.

Referring to FIG. 15, a first transducer 300 and a second transducer 400 may be prepared. The first and second transducers 300 and 400 may correspond to the first and second transducers 40 and 42, or the third and fourth transducers 46 and 48, in FIG. 1. Also, the first and second transducers 300 and 400 may correspond to the first and second transducers 84 and 86, or the third and fourth transducers 94 and 96, in FIG. 6.

The first transducer 300 may include an ultrasonic wave transmitter/receiver 302 and a first connector 304. A first through hole 304h may be formed in the first connector 304. The second transducer 400 may include an ultrasonic wave transmitter/receiver 402 and a second connector 404. A second through hole 404h may be formed in the second connector 404.

Then, as shown in FIG. 16, the first through hole 304h in the first connector 304 of the first transducer 300 and the second through hole 404h in the second connector 404 of the second transducer 400 may be aligned to insert the second connector 404 of the second transducer 400 into the first connector 304 of the first transducer 300. Next, a connecting member 44 may be inserted into the first and second through holes 304h and 404h. The connecting member 44 may be a member that may rotatably connect the first and second connectors 304 and 404. The connecting member 44 may be, for example, a pin, a hinge, an actuator, or the like. In this way, the first and second transducers 300 and 400 that may be rotatably connected to each other may be manufactured. In the same way, the third and the fourth transducer (not shown) that may be rotatably connected to each other may be manufactured. The transducers manufactured in the aforementioned way may be symmetrically disposed around the subject 110 in the case 150, as shown in FIG. 11.

In FIG. 6, the first and second transducers 84 and 86, and the third and fourth transducers 94 and 96 may be manufactured by the above method to form the transducer 100. When preparing the first and the third transducers 84 and 94, elements described by referring to FIGS. 7 to 10 may be formed in the sliding portions 82 and 92 of the first and the third transducers 84 and 94. Then, the sliding portion 82 of the first transducer 84 may be connected to the sliding portion 92 of the second transducer 94. The connection between the first and second transducers 84 and 86, and the connection between the third and fourth transducers 94 and 96 may be performed after the sliding portion 82 of the first transducer 84 is connected to the sliding portion 92 of the second transducer 94.

As described above, according to one or more of the above example embodiments, more transducers may transmit and receive ultrasonic signals compared to an existing method in which transducers at one side transmit or receive ultrasonic signals. In addition, the scanning data in at least two directions may be obtained without an unnecessary rotation of the transducer, and thus a vortex in a liquid may be prevented. As a result, the degradation of the quality of the subject image caused by the vortex may be prevented and the scanning time may be shortened.

Additionally, the test is conducted in at least two directions rather than in one direction, and thus more data is obtained and more accurate images are produced based on the data.

The units, controllers, and/or modules described herein may be implemented using hardware components, software components, or a combination thereof. For example, the hardware components may include microcontrollers, memory modules, sensors, microphones, amplifiers, band-pass filters, audio to digital converters, and processing devices, or the like. A processing device may be implemented using one or more hardware device configured to carry out and/or execute program code by performing arithmetical, logical, and input/output operations. The processing device(s) may include a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such a parallel processors.

The software may include a computer program, a piece of code, an instruction, or some combination thereof, to independently or collectively instruct and/or configure the processing device to operate as desired, thereby transforming the processing device into a special purpose processor. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. The software and data may be stored by one or more non-transitory computer readable recording mediums.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of some example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher level code that may be executed by the computer using an interpreter. The above-described devices may be configured to act as one or more software modules in order to perform the operations of the above-described example embodiments, or vice versa.

It should be understood that example embodiments described herein should be considered in a descriptive sense only and not for purposes of limitation. Descriptions of features or aspects within each device or method according to example embodiments should typically be considered as available for other similar features or aspects in other devices or methods according to example embodiments. While some example embodiments have been particularly shown and described, it will be understood by one of ordinary skill in the art that variations in form and detail may be made therein without departing from the spirit and scope of the claims.

What is claimed is:

1. An ultrasonic wave diagnosis apparatus comprising:
a first transducer unit including a plurality of transducers, the first transducer unit configured to emit ultrasonic waves towards a subject; and
a second transducer unit including a plurality of transducers, the second transducer unit configured to receive the ultrasonic waves emitted by the first transducer unit.

2. The ultrasonic wave diagnosis apparatus of claim 1, wherein the first transducer unit and the second transducer unit are configured to be symmetrically placed around the subject.

3. The ultrasonic wave diagnosis apparatus of claim 1, wherein the first transducer unit and the second transducer unit are configured to be connected to each other.

4. The ultrasonic wave diagnosis apparatus of claim 3, wherein the first transducer unit and the second transducer unit are configured to be connected to each other in a sliding manner.

5. The ultrasonic wave diagnosis apparatus of claim 1, wherein the first transducer unit includes:
a first transducer configured to emit first ultrasonic waves towards the subject;
a second transducer configured to emit second ultrasonic waves towards the subject; and
a connecting member configured to connect the first transducer to the second transducer.

6. The ultrasonic wave diagnosis apparatus of claim 1, wherein the second transducer unit includes:
a first transducer configured to receive first ultrasonic waves emitted by the first transducer unit;
a second transducer configured to receive first ultrasonic waves emitted by the first transducer unit; and
a connecting member configured to connect the first transducer to second transducer.

7. A method of manufacturing an ultrasonic wave diagnosis apparatus, the method comprising:
preparing a first transducer that comprises a first ultrasonic wave transmitter/receiver and a first connector;
preparing a second transducer that comprises a second ultrasonic wave transmitter/receiver and a second connector; and
connecting the first connector to the second connector, thereby forming a first transducer unit of a ultrasonic wave diagnosis apparatus.

8. The method of claim 7, further comprising:
preparing a third transducer that comprises a third ultrasonic wave transmitter/receiver and a third connector;
preparing a fourth transducer that comprises a fourth ultrasonic wave transmitter/receiver and a fourth connector; and
connecting the third connector to the fourth connector, thereby forming a second transducer unit of the ultrasonic wave diagnosis apparatus.

9. The method of claim 7, wherein the preparing of the first transducer includes forming a first through hole in the first connector.

10. The method of claim 7, wherein the preparing of the second transducer includes forming a second through hole in the second connector.

11. The method of claim 7, wherein the connecting of the first and second connectors includes inserting a connecting member, which passes through the first and second connectors, into the first and second connectors.

12. The method of claim 8, wherein the preparing of the third transducer includes forming a third through hole in the third connector.

13. The method of claim 8, wherein the preparing of the fourth transducer includes forming a fourth through hole in the fourth connector.

14. The method of claim 8, wherein the connecting of the third and fourth connectors includes inserting a connecting member, which passes through the third and fourth connectors, into the third and fourth connectors.

15. The method of claim 8, wherein the first transducer further includes a first sliding portion, and the third transducer further includes a second sliding portion.

16. The method of claim 15, wherein elements required for connecting the first sliding portion to the second sliding portion are formed in the first sliding portion before connecting the first and second transducers to each other, and
elements for the connection with the first sliding portion are formed in the second sliding portion before connecting the third and fourth transducers to each other.

17. The method of claim 16, wherein the first sliding portion and the second sliding portion are connected to each other after the first and second transducers are connected to each other and after the third and fourth transducers are connected to each other.

18. The method of claim 16, wherein the first sliding portion and the second sliding portion are connected to each other, and then the first and second transducers are connected to each other and the third and fourth transducers are connected to each other.
